(19) United States
(12) Patent Application Publication
(10) Pub. No.: US 2022/0330848 A1
(43) Pub. Date: Oct. 20, 2022

(54) Method, Computer Program, and Device for Determining Vehicle Occupant Respiration

(71) Applicant: Bayerische Motoren Werke Aktiengesellschaft, Muenchen (DE)

(72) Inventors: Noel Ferraris, Mountain View, CA (US); Etienne Iliffe-Moon, Menlo Park, CA (US); Anderson Vankalaya, San Francisco, CA (US)

(21) Appl. No.: 17/232,172

(22) Filed: Apr. 16, 2021

(51) Int. Cl. A61B 5/08; B60W 40/08; G06K 9/00; G06K 9/62; H04R 1/10; H04R 1/32; G06N 3/08

(52) U.S. Cl. CPC A61B 5/0816; B60W 40/08; G06K 9/00832; G06K 9/00288; G06K 9/6289; H04R 1/1083; H04R 1/326; G06N 3/08; B60W 2540/01; B60W 2540/221; H04R 2410/01; H04R 2499/13

(57) ABSTRACT

A vehicular device is disclosed, including a processor and plurality of sensors. The processor receives sensor data and determines vehicle occupant respiration based on the sensor data. The sensors can include acoustic sensors and imaging sensors. The processor can be configured for executing a machine learning algorithm to determine the occupant respiration.

### FIELD

Embodiments relate to a method, computer program, and device for determining occupant respiration in a vehicle.

### BACKGROUND

It is a goal to continually improve the pleasure of driving. Vehicles have environmental and/or performance controls that may be adjusted to increase driver comfort, e.g by adjusting cabin temperature. Drivers (and passengers) attitudes toward the driving experience and cabin environment may change during a trip such that it may be desirable to tweak environmental and/or performance to increase driver comfort or pleasure. It can be challenging to determine or predict the environmental and/or performance adjustments that are desirable to vehicle occupants, such things being dependent on driver's attitude, state, or condition. Herein is disclosed a device and method for determining vehicle occupant respiration, which may be used as a basis for adjusting vehicle performance and/or cabin environment, for example.

### SUMMARY

Disclosed herein is a vehicular device for determining occupant respiration. The device can include a processor which receives sensor data and determines occupant respiration based on the sensor data. A plurality of sensors may transmit sensor data to the processor. In order to increase the pleasure of driving, it may be possible to make adjustments such as to the cabin environment and vehicle performance based on the state of the driver. It may be possible to improve the driving experience by better understanding the state of the driver, such as through a determination of vehicle occupants' respiration.

### BRIEF DESCRIPTION OF THE FIGURES

Some examples of apparatuses and/or methods will be described in the following by way of example only, and with reference to the accompanying figures.

FIG. 1 shows a schematic of a vehicular device, according to embodiments described herein.

FIG. 2 shows a block diagram of a method of determining vehicle occupant respiration, according to embodiments described herein.

FIG. 3 shows a schematic of a vehicular device, according to embodiments described herein.

### DETAILED DESCRIPTION

Some examples are now described in more detail with reference to the enclosed figures. However, other possible examples are not limited to the features of these embodiments described in detail. Other examples may include modifications of the features as well as equivalents and alternatives to the features. Furthermore, the terminology used herein to describe certain examples should not be restrictive of further possible examples.

Throughout the description of the figures same or similar reference numerals refer to same or similar elements and/or features, which may be identical or implemented in a modified form while providing the same or a similar function. The thickness of lines, layers and/or areas in the figures may also be exaggerated for clarification.

When two elements A and B are combined using an ‘or’, this is to be understood as disclosing all possible combinations, i.e. only A, only B as well as A and B, unless expressly defined otherwise in the individual case. As an alternative wording for the same combinations, “at least one of A and B” or “A and/or B” may be used. This applies equivalently to combinations of more than two elements.

If a singular form, such as “a”, “an” and “the” is used and the use of only a single element is not defined as mandatory either explicitly or implicitly, further examples may also use several elements to implement the same function. If a function is described below as implemented using multiple elements, further examples may implement the same function using a single element or a single processing entity. It is further understood that the terms “include”, “including”, “comprise” and/or “comprising”, when used, describe the presence of the specified features, integers, steps, operations, processes, elements, components and/or a group thereof, but do not necessarily exclude the presence or addition of one or more other features, integers, steps, operations, processes, elements, components and/or a group thereof.

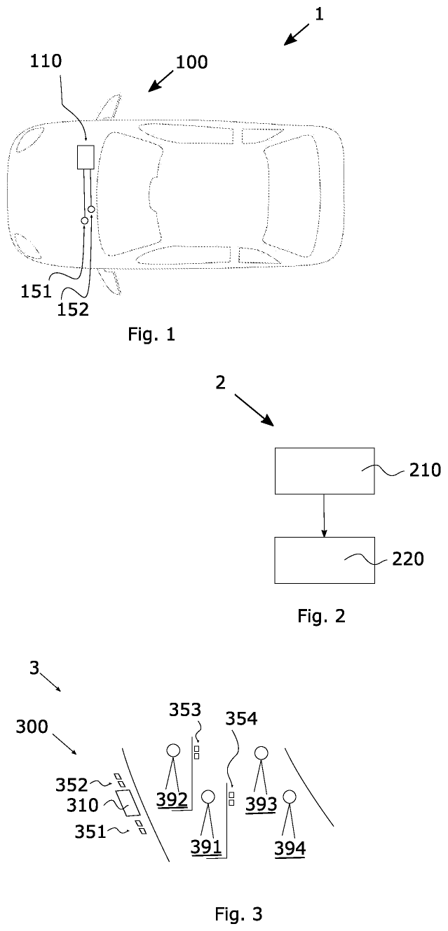

FIG. 1 shows a schematic of a vehicular device, according to embodiments described herein, such as is described with respect to other figures and/or embodiments disclosed herein. The vehicular device 100 includes a processor 110 which is communicatively coupled to at least one sensor, such as two or more sensors 151, 152. The processor 110 can receive sensor data from the sensor(s). The processor 110 can be programmed to determine the respiration of an occupant of the vehicle 1, e.g. the respiration of a driver and/or passenger(s). The determination may provide dynamic parameters that may model the occupant's respiration, e.g. based on sensor data. The respiration determination can include respiration rate, for example. The respiratory phase can alternatively/additionally be determined, e.g. dynamically. The sensor(s) may provide data which allows for the determination of at least one respiration parameter, which may include at least one of: respiration rate; respiration amplitude; and respiration phase. Respiration phase can include inhalation, exhalation, and transitions therebetween, e.g from inhaling to exhaling or from exhaling to inhaling.

The respiration determination may utilize real-time determinations, e.g. based on real-time data determinations and analysis. The respiration determination may include a time based determination.

The occupant respiration, as determined, may trigger and/or modify environmental and/or performance controls of the vehicle. For example, a determination of the occupant respiration and/or classification of the occupant respiration may trigger any of: a lighting change, an audio output change (e.g a change of music), a change of exhaust note such as to alter volume and/or pitch, a change in the seating geometry (e.g. effecting a change in posture of the occupant), suspension characteristics (soften or harden the suspension), opening/closing the moonroof or sunroof, a seat heater, a seat massage unit, temperature adjustment, visor adjustment, window adjustment, and combinations thereof. Alternative/additionally, the occupant respiration may trigger an alarm such as to alert the occupant(s). In another example, the driver's respiration may be used as a basis for determining the driver' s attention. For example the respiration may be a basis for determining a change into or out of autonomous driving mode; e.g. the performance control may be switched from autonomous control to driver control. Alternatively/additionally, the respiration determination may trigger variable adjustments to the brightness and/or color of ambient lighting in the cabin, and/or airflow in the cabin. The environmental and/or performance control modifications may express the respiration state of the occupant, e.g. as a wellness or meditation experience.

The determination of the occupant's respiration may be used to increase safety and/or the alter the experience of driving, such as to stimulate or comfort the occupant(s), e.g to increase awareness or reduce physical fatigue. Such changes, due to the determination of occupant respiration, may be executed to any number of the vehicle occupants, such as to all the occupants, only the driver, or only the passenger(s). It is desirable for the vehicle to make desirable changes, e.g environmental and/or performance changes, without requiring operator action, e.g without requiring active input from any of the vehicle's occupants. It is believed that the respiration determination of vehicle occupants may be a useful metric on which technical adjustments that impact the driving experience can be made. The data used for determining respiration can be combined with additional data, e.g contextual data, to even further improve the model of the driver's state. Alternatively/additionally, the combination of additional data with the sensor data for determining respiration may better inform the adjustment of performance and/or environmental controls.

Herein is disclosed various configurations of a device for determining occupant respiration. It is particularly desirable to have nonintrusive configurations such as configurations which make minimal or no contact with the occupants. For example, determining respiration can involve sensing heart rate variability and possibly acquiring electrocardiogram data. A wearable device and/or device making contact with the body may be required for determining such data. It may be challenging to determine respiration using noncontact means. Another challenge is to have low latency in the respiration determination. Yet another challenge is accuracy in the respiration determination, particularly in the presence of various sources of sensor noise and possible intermittent loss of signal.

Returning to FIG. 1, the sensors 151, 152 can include at least one acoustic sensor 151, which may include a directional microphone. The acoustic sensor(s) 151 may include sensor(s) at the instrument panel and/or the steering wheel. The acoustic sensor(s) may pick up audio to determine breathing. For example, a directional microphone may be directed at the mouth and/or nose of an occupant. Multiple acoustic sensor(s) may be used, such as to increase signal and/or provide signals for performing noise cancellation, e.g. with a noise filter which may be part of the processor 110.

In an embodiment, the sensors, including the acoustic sensor(s), are remote sensors, e.g. the sensors are not worn by the occupants. Remote sensors can be desirable, e.g. to provide an unintrusive means of acquiring the data for determination of the respiration of the occupant(s). For example, at least one of the sensors can be a sensor in the seat, such as an inertial sensor and/or an audio sensor.

It is contemplated to use a wearable device such as a headset, e.g. a wireless and/or Bluetooth headset, for acquiring acoustic data, particularly when an occupant is connected to an onboard communication system, e.g. a system for wireless communication.

The processor 110 can be configured for parallel execution of: classifying an audio signal based on the audio sensor data as inhalation, exhalation, or ambience; and determining a transition of exhalation and inhalation. The audio signal used for the classification can be based on the audio sensor data from at least one of the audio sensors. The audio signal may be preprocessed such as noise filtered.

The sensor(s) 151, 152 of the device 100 can include at least one camera 152, such as camera(s) for determining thermal data and/or visible light data, particularly of the facial region of an occupant. The device can include optics which collect visible and/or infrared radiation from the facial region of an occupant. Camera(s) that are capable of providing visible image data and thermal data from the same region, e.g. the facial region of an occupant, may provide particularly relevant data for determining respiration. For example, data can include red, green, blue (RGB) data and thermal and/or infrared data.

For example, captured images can include a channel of data, e.g. thermal data, for that corresponds to the temperature perceived by the camera(s), e.g. for every pixel of the visible image data, there is also a temperature and/or infrared channel or pixel. The images may also include RGB channels that can correspond to the visible image data.

The processor can determine a facial region of an occupant, e.g. based at least in part on the visible light data. For example, the processor can determine/apply a bounding box (e.g. to the image data) based on the facial region. The bounding box may have a quaternion format, which may be particularly convenient considering the possible movement of the occupant's face. Determination of the facial region may make even further determinations to be made.

For example, the determination of the facial region, and/or the determination of the mouth and noise region, can be a basis for determining a target direction of the directional microphone.

Alternatively/additionally, the determination of the facial region, and/or the determination of the mouth and noise region, can be through the visible data, and this may allow for the corresponding region of the thermal data (e.g thermal image data) acquired from the camera(s) to be determined. For example, when thermal data and visible light data are collected from array sensors, the visible light data can be used to determine the facial region, and the corresponding region of the thermal image sensor array (e.g. an infrared sensor array) can be determined.

The data from the visible sensor array, e.g. the RGB channels, can be passed to an algorithmic model such as a neural network (such as a convolutional neural net) that can localize (e.g. using object detection), segment, and/or identify the facial region, such as any number of facial features such has the eyes, nose, and lips of an occupant. The data from the visible sensor array can used to identify the pixels that correspond to the air breathed in/out. The facial data can be segmented to identify such pixels.

The algorithmic model can be trained with occlusion, e.g. intermittent blocking of the line of sight from the visible sensor array to the facial region. The model can be trained with varied levels of natural and artificial lighting in a vehicle setting. It is possible to determine the facial region of a target even in the presence of multiple intermittent faces, e.g. intermittently sensed faces that are not the target for determination of respiration.

Bounding boxes that can be determined/generated can be in a quaternion format, e.g to account for rotation of the human face. The corresponding pixel values can be extracted from the thermal/infrared/temperature channel. A matrix corresponding to thermal data can be aggregated and/or averaged over time. At least one noise filtering algorithm can be applied.

The processor may be programmed to determine if the air is being inhaled, exhaled, or transitioning from inhalation to exhalation, or vice versa. An algorithm may pool the thermal data captured by the camera within a time window and/or at a region away from the occupant(s), such as to determine ambient temperature. The ambient temperature determination can be compared with a matrix corresponding to the thermal data of the facial region, such as at the air underneath the nose of the occupant.

It is particularly contemplated to pool at least thermal and audio data to determine the respiration, e.g in a sensor fusion algorithm. Additional sources of data, such as visible light data may also be pooled. Pooling of data from different sources may increase accuracy of the respiration determination. For example, multiple data sources may allow weighting of the data sources to change over time, which can compensate for intermittent signal drops, or intermittent noise in some channels of data by providing alternative channels of data. In an example, the cabin noise floor may be too high for accurate determination of respiration by one or more microphones; in such a case, there may be alternative sources of data, e.g. from other sensors (using thermal data, visible light data, and/or data from other microphones), that may allow for determination of the respiration.

The processor can be configured for parallel execution of: classifying a signal based on the sensor data as inhalation, exhalation, or ambience; and determining a transition of exhalation and inhalation. For example, the processor can be configured for parallel execution of: classifying a thermal signal based on the thermal camera data as inhalation, exhalation, or ambience; and determining a transition of exhalation and inhalation. For example, the processor can be configured for parallel execution of: classifying a thermal signal based on the thermal camera data as inhalation, exhalation, or ambience; classifying an acoustic signal based on the acoustic data as inhalation, exhalation, or ambience; and determining a transition of exhalation and inhalation.

Alternatively/additionally, the processor can be configured for parallel execution of: classifying a hybrid signal or combination of signals based on the camera data (e.g. at least the thermal camera data) and acoustic data as inhalation, exhalation, or ambience; and determining a transition of exhalation and inhalation.

The processor 110 may be an on-board processor. An onboard processor, e.g. one that is present in the vehicle rather than remotely communicatively coupled to the vehicle, such as a cloud device, may reduce latency. An onboard processor may also provide for greater bandwidth and/or privacy for the occupant(s) in comparison to a cloud based processor(s). An on-board processor may also reduce power consumption. It is particularly contemplated to have a processor(s) on board which has multiple-thread capability, e.g. for parallel execution. Alternatively/additionally, the processor 110 can be an edge device, such as a processor with the capability of receiving and/or transmitting data with nearby vehicles. Data received from other vehicles may be used in combination with the sensor data and/or respiration determination, e.g. in making environmental/performance changes to the vehicle 1. An on-board edge computer is particularly contemplated as the processor, such as one that performs the respiration determination using sensors within the occupant's vehicle. An on-board processor, such as an on-board edge processor, could be programmed for the capability of making environmental/performance adjustments based at least partially on the respiration determination and optionally based additionally on additional data, such as data received from nearby vehicles, other edge computing nodes, and/or the cloud.

The processor 110 may be an on-board processor that is communicatively couplable to an external device and/or the cloud. For example, a network and/or the cloud may be used to patch and/or update the software, such as the models/algorithms, e.g to increase accuracy. The device can be configured for communication such that local user data is kept strictly on-board (e.g. with a possible exception being that the user(s) has explicitly given permission). For example, sensor data is kept on-board and/or not provided to any external device such as a network, cloud, other edge devices or edge nodes. Such strict control over data usage may be desirable for user privacy concerns.

The process may determine the respiration by a sensor fusion machine learning algorithm, for example. The sensor fusion algorithm may be an ensemble learning based artificial neural network.

These processor methods, such as sensor fusion machine learning can use and/or combine with an artificial neural network (ANN). The inputs to the ANN can be the occupant respiration as determined, e.g. the classification of exhalation, inhalation, and transitions. The inputs to the ANN can also be probability strengths from audio and the image models based respectively on the acoustic and camera sensors. Alternatively/additionally, the inputs to the ANN can be used to classify the respiration state of the occupant(s). For example, the respiration state can be determined as classified according to a plurality of possible states. For example, the states may include levels of alertness and levels of comfort, e.g the respiration state is modeled as an array of parameters. Alternatively/additionally, the states may be a set of determined vectors (e.g. sets of parameters) which are determined by machine learning algorithm.

The respiration determination may include a respiration state determination. For example, the sensor(s) data can be used as a basis for determination of the respiration state (the sensor(s) data can be the direct basis for the determination of the respiration state). In another example, the dynamic parameters determined to model the occupant(s) respiration may be used as a basis for the respiration state determination.

It is particularly contemplated to use machine learning such as a classification algorithm and/or principal component analysis for respiration state determination.

The processor 110, and/or sensor fusion machine learning algorithm, may allow for determination of the respiration rate even if data is intermittently missing from one or more of the sensors. One or more of the sensors may go off-line, or fail, or the like. In another example, the facial region may be occluded, e.g. by a hand. The sensor fusion machine learning algorithm can continue to determine respiration when one or more of the pipelines, e.g. data inputs/streams from the sensor(s), is paused, lost, and/or fails. For example, a vision pipeline might not detect a person and/or facial region if lighting conditions are outside a tolerance. For example, a vision pipeline might fail if the person's face is oriented in a way that the nose is occluded (or partially occluded). The ANN may provide an output as the final output of the model which is used to drive the business logic/use-case, e.g. to determine the changes in environmental/performance control of the vehicle.

The sensor fusion approach algorithm may be trained over a period of time, such as starting from before initial ownership, starting from a time of initial vehicle ownership, or over a longer period of time. The system may be adaptable to correlate and/or combine occupant respiration information with contextual data/information from other vehicle systems. The contextual data may be diverse, for example, at least one of: calendar, location, traffic, day/time, driver attention, stress, emotion, or heart rate.

The sensor fusion algorithm can be trained with audio data, such as using a dataset of audio data that is labeled such that the respiration is already know, e.g. the phase and amplitude of the respiration.

The sensor fusion input can include at least one of: acoustic data (which may be noise filtered and/or directional), thermal data (e.g. air temperature), or visible light data. The acoustic data may come from one or more acoustic sensors. The acoustic data can be down-sampled, e.g. from a typical input frequency of 44.1 kHz, e.g. in order to reduce the computational burden of the data processing and/or reduce noise in the audio waveform. The down-sampling can be done without significantly modifying the original source. The thermal data may come from one or more thermal sensors, such as one or more cameras sensitive to infrared. The visible light data may come from one or more visible light sensors, such as one or more cameras.

The sensors 151, 152 may communicate with the processor 110 in real-time, e.g. transmitting data to repeatedly update the determination of the respiration. Significant changes to the respiration determination may trigger environmental/performance changes of the vehicle. Alternatively/additionally, low variation in the respiration determination over a duration may trigger environmental/performance changes. Respiration may be one of a plurality of determined parameters for inducing environmental/performance changes. For example, contextual data may be used in combination with the respiration determination, and/or sensor data for determining respiration, for making changes to environmental/performance controls of the vehicle.

The methods described herein may improve driving safety, for example, by reducing the interaction of driver with environmental/performance controls. A machine learning algorithm can be trained, and/or have as the learning objective, to reduce driver interaction with environmental controls, to minimize driver distraction, and/or maximize occupant(s) comfort. For example, the respiration determination may be correlated with occupant's adjustments of the environmental/performance controls, and the machine learning algorithm trained to predict such adjustments based on the respiration.

The sensors may include a sensor which is capable of detecting the expansion and/or contraction of the chest region. For example, at least one depth camera can be used. The respiration determination can include determining the volume of air inhaled and/or exhaled.

FIG. 2 illustrates a method of determining vehicle occupant respiration, according to embodiments described herein, such as is described with respect to other figures and/or embodiments disclosed herein, particularly FIG. 1 above and/or the above description.

The method 2 includes acquiring 210 sensor data from a plurality of sensors in a vehicle, and determining 220 occupant respiration based on the sensor data. Determining 220 the occupant respiration can include determining at least one of: respiration rate; respiration amplitude; and respiration phase which includes inhalation, exhalation, and transitions therebetween.

The sensor data can include audio sensor and/or imaging sensor data. For example, in determining the respiration, an audio signal can be classified, based on audio sensor data of the sensor data, the classification being as inhalation, exhalation, or ambience. The respiration determination can include determining a transition of exhalation and inhalation. It is possible to execute the classification of phase (e.g. inhalation, exhalation) and the determination of the transition in parallel, e.g. using a multithread processor. A multithread processor may allow keeping pace with the computational load.

In determining the respiration, the facial region of an occupant based on visible light data of the sensor data can be determined. A bounding box based on the facial region can be determined, e.g. the bounding box having a quaternion format. The occupant respiration can be determined based on thermal camera data at the facial region, for example. Alternatively/additionally, the operation of a directional microphone for picking up audio from the mouth and nose region of the occupant can be determined based on the identification/determination of the facial region.

The occupant respiration can be determined based on executing sensor fusion machine learning, e.g based on sensor fusion input. The sensor fusion input can include at least one of: acoustic data, thermal data, or visible light data.

A non-transitory computer readable medium can include instructions adapted to determine vehicle occupant respiration, using the methods described herein, and/or using the device as described herein.

Herein “ambience” may refer to an absent acoustic signal, background acoustic signal, unidentifiable acoustic signal, and/or acoustic signal that may not directly impact the respiration determination, e.g. is ignored in the data processing. Herein a directional microphone may refer to a microphone with a greater sensitivity in a particular direction; alternatively/additionally a directional microphone may be adjustable to adjust the position of maximum sensitivity.

Herein, a trailing “(s)” or “(es)” indicates an optional plurality. For example, “processor(s)” means “one or more processor,” “at least one processor,” or “a processor and optionally more processors.” Herein a slash “/” indicates “and/or” which conveys “‘and’ or ‘or’”. Thus “A/B” means “A and/or B;” equivalently, “A/B” means: an A alone, a B alone, and an A and a B;

equivalently “at least one of A and B.”

The device 100 can include a receiver and/or transmitter, or can interface with a receiver and/or transmitter for the communication of data, for example between the processor 110 and the sensor(s) 151, 152 and/or other vehicles. For example, the device 100 can include a means for obtaining, receiving, transmitting or providing analog or digital signals or information, e.g. any connector, contact, pin, register, input port, output port, conductor, lane, etc. which allows providing or obtaining a signal or information. The device 100 can communicate data with internal or external components, for example. The device 100 can communicate and/or include components to enable communication, such as a mobile communication system.

The processor 110 described herein may alternatively be a plurality of processors. The methods described herein may be performed by a processor and/or plurality of processors. One or more processing units can be any means for processing, such as a processor, a computer or a programmable hardware component operable with accordingly adapted software. The methods described herein may be implemented in software, such as software executed on one or more programmable hardware components. Such hardware components may comprise a general-purpose processor, a Digital Signal Processor (DSP), a micro-controller, etc.

Herein to “pool” can mean to combine data. For example, audio data, thermal data, and visible light data can be pooled and used as input in an algorithm, such as a machine learning algorithm for determining respiration.

In an embodiment the device 100 may include a memory and a processor(s) 110 operably coupled to the memory and configured to perform the methods described herein.

FIG. 3 illustrates a schematic of a vehicular device, according to embodiments described herein, such as is described with respect to other figures and/or embodiments disclosed herein.

The vehicular device 300 includes a processor 310 which is communicatively coupled to at least one sensor, such as sensors 351, 352, 353, 354. The processor 310 can receive sensor data from the sensor(s). The processor 310 can be programmed to determine the respiration of an occupant of the vehicle 3, e.g. the respiration of a driver 391 and/or passenger(s) 392, 393, 394. The sensors 351, 352, 353, 354 can sense multiple occupants of the vehicle 3. For example, a set of sensors 351 may be configured to determine data from one occupant, such as the driver 391. A second set of sensors 352 may be configured to determine data from the front passenger 392. There may be sets of sensors 353, 354 for determining data from the backseat passengers 393, 394 individually. Alternatively/additionally, at least one sensor may not be dedicated to a single passenger, such as sensor(s) for noise cancellation, e.g audio noise that may be common to a varying extent to all microphones.

The aspects and features described in relation to a particular one of the previous examples may also be combined with one or more of the further examples to replace an identical or similar feature of that further example or to additionally introduce the features into the further example.

Examples may further be or relate to a (computer) program including a program code to execute one or more of the methods described herein when the program is executed on a computer, processor or other programmable hardware component. Steps, operations or processes of the methods described herein may be executed by programmed computers, processors or other programmable hardware components. Examples may also cover program storage devices, such as digital data storage media, which are machine-, processor- or computer-readable and encode and/or contain machine-executable, processor-executable or computer-executable programs and instructions. Program storage devices may include or be digital storage devices, magnetic storage media such as magnetic disks and magnetic tapes, hard disk drives, or optically readable digital data storage media, for example. Other examples may also include computers, processors, control units, (field) programmable logic arrays ((F)PLAs), (field) programmable gate arrays ((F)PGAs), graphics processor units (GPU), application-specific integrated circuits (ASICs), integrated circuits (ICs) or system-on-a-chip (SoCs) systems programmed to execute the steps of the methods described above.

It is further understood that the disclosure of several steps, processes, operations or functions disclosed in the description or claims shall not be construed to imply that these operations are necessarily dependent on the order described, unless explicitly stated in the individual case or necessary for technical reasons. Therefore, the previous description does not limit the execution of several steps or functions to a certain order. Furthermore, in further examples, a single step, function, process or operation may include and/or be broken up into several sub-steps, -functions, -processes or -operations.

If some aspects have been described in relation to a device or system, these aspects should also be understood as a description of the corresponding method. For example, a block, device or functional aspect of the device or system may correspond to a feature, such as a method step, of the corresponding method. Accordingly, aspects described in relation to a method shall also be understood as a description of a corresponding block, a corresponding element, a property or a functional feature of a corresponding device or a corresponding system.

If some aspects have been described in relation to a device or system, these aspects should also be understood as a description of the corresponding method and vice versa. For example, a block, device or functional aspect of the device or system may correspond to a feature, such as a method step, of the corresponding method. Accordingly, aspects described in relation to a method shall also be understood as a description of a corresponding block, a corresponding element, a property or a functional feature of a corresponding device or a corresponding system.

Herein, machine learning can refer to algorithms and/or statistical models that computer systems may use to perform tasks such as to determine respiration. Machine learning may possibly forgo the use of particularized instructions, instead utilizing models and inference.

For example, in machine-learning, instead of a rule-based transformation of data, a transformation of data may be used that is inferred from an analysis of historical and/or training data. For example, sensor data may be analyzed using a machine-learning model or using a machine-learning algorithm.

In order for the machine-learning model to analyze the sensor data, the machine-learning model may be trained using training data as input and training information. By training the machine-learning model with a large dataset of sensor data as training content information, the machine-learning model “learns” to recognize the sensor data, e.g. learns to determine the respiration based on limited sensor data by taking advantage of training data which may include more data, e.g. data also from sensors that provide highly accurate and high signal-to-noise respiratory related data. The respiration can be determined even when data which is not directly included in the training data can be utilized and/or recognized using the machine-learning model. By training a machine-learning model using training sensor data and a desired output (e.g. known respiration parameters), the machine-learning model can learn.

For example, a respiratory sensor data can be used for training, possibly including sensors in contact with a vehicle occupant. Such a respiratory sensor in the device for on-board use may be undesirable due to cost and/or invasiveness of the sensor and method. Nevertheless, such a respiratory sensor(s) may be used to train the model to utilize the data from less invasive sensors, such as microphones and cameras.

Machine-learning models can be trained using training input data. Supervised learning can be used. In supervised learning, the machine-learning model can be trained using a plurality of training samples, wherein each sample may include a plurality of input data values, and a plurality of desired output values, i.e. each training sample is associated with a desired output value. By specifying both training samples and desired output values, the machine-learning model “learns” which output value to provide based on an input sample that is similar to the samples provided during the training.

Semi-supervised learning may be used. In semi-supervised learning, some of the training samples may lack a corresponding desired output value. Supervised learning may be based on a supervised learning algorithm, e.g. a classification algorithm, a regression algorithm or a similarity learning algorithm. Classification algorithms may be used when the outputs are restricted to a limited set of values, i.e. the input is classified to one of the limited set of values. Regression algorithms may be used when the outputs may have any numerical value (within a range). Similarity learning algorithms are similar to both classification and regression algorithms. Similarity learning algorithms may be based on learning from examples using a similarity function that measures how similar or related two objects, e.g. sets of sensor data, are.

Apart from supervised or semi-supervised learning, unsupervised learning may be used to train the machine-learning model. In unsupervised learning, (only) input data might be supplied, and an unsupervised learning algorithm may be used to find structure in the input data, e.g. by grouping or clustering the input data, finding commonalities in the data. Clustering is the assignment of input data comprising a plurality of input values into subsets (clusters) so that input values within the same cluster are similar according to one or more (pre-defined) similarity criteria, while being dissimilar to input values that are included in other clusters.

Reinforcement learning may be used alternatively/additionally. Reinforcement learning may be used to train the machine-learning model. In reinforcement learning, one or more software actors (called “software agents”) are trained to take actions in an environment. Based on the taken actions, a reward is calculated. Reinforcement learning is based on training the one or more software agents to choose the actions such that the cumulative reward is increased, leading to software agents that become better at the task they are given (as evidenced by increasing rewards).

Furthermore, some techniques may be applied to some of the machine-learning algorithms. For example, feature learning may be used. In other words, the machine-learning model may at least partially be trained using feature learning, and/or the machine-learning algorithm may comprise a feature learning component. Feature learning algorithms, which may be called representation learning algorithms, may preserve the information in their input, but also transform it in a way that makes it useful, often as a pre-processing step before performing classification or predictions. Feature learning may be based on principal components analysis or cluster analysis, for example.

In some examples, anomaly detection (i.e. outlier detection) may be used, which is aimed at providing an identification of input values that raise suspicions by differing significantly from the majority of input or training data. In other words, the machine-learning model may at least partially be trained using anomaly detection, and/or the machine-learning algorithm may comprise an anomaly detection component. Occlusion detection as mentioned herein may be a type of anomaly detection.

In some examples, the machine-learning algorithm may use a decision tree as a predictive model. In other words, the machine-learning model may be based on a decision tree. In a decision tree, observations about an item (e.g. a set of sensor data) may be represented by the branches of the decision tree, and an output value corresponding to the item may be represented by the leaves of the decision tree. Decision trees may support both discrete values and continuous values as output values. If discrete values are used, the decision tree may be denoted a classification tree, if continuous values are used, the decision tree may be denoted a regression tree.

Association rules may be used in machine-learning algorithms. In other words, the machine-learning model may be based on one or more association rules. Association rules can be created by identifying relationships between variables in large amounts of data. The machine-learning algorithm may identify and/or utilize one or more relational rules that represent the knowledge that is derived from the data. The rules may e.g. be used to store, manipulate or apply the knowledge.

Machine-learning algorithms are usually based on a machine-learning model. The term “machine-learning algorithm” may denote a set of instructions that may be used to create, train, or use a machine-learning model. The term “machine-learning model” may denote a data structure and/or set of rules that represents the learned knowledge, e.g. based on the training performed by the machine-learning algorithm. In embodiments, the usage of a machine-learning algorithm may imply the usage of an underlying machine-learning model (or of a plurality of underlying machine-learning models). The usage of a machine-learning model may imply that the machine-learning model and/or the data structure/set of rules that is the machine-learning model is trained by a machine-learning algorithm.

For example, the machine-learning model may be an artificial neural network (ANN). ANNs are systems that are inspired by biological neural networks, such as can be found in a brain. ANNs comprise a plurality of interconnected nodes and a plurality of connections, so-called edges, between the nodes. There are usually three types of nodes, input nodes that receive input values, hidden nodes that are (only) connected to other nodes, and output nodes that provide output values. Each node may represent an artificial neuron. Each edge may transmit information, from one node to another. The output of a node may be defined as a (non-linear) function of the sum of its inputs. The inputs of a node may be used in the function based on a “weight” of the edge or of the node that provides the input. The weight of nodes and/or of edges may be adjusted in the learning process. In other words, the training of an artificial neural network may comprise adjusting the weights of the nodes and/or edges of the artificial neural network, i.e. to achieve a desired output for a given input. In at least some embodiments, the machine-learning model may be deep neural network, e.g. a neural network comprising one or more layers of hidden nodes (i.e. hidden layers), prefer-ably a plurality of layers of hidden nodes.

Alternatively, the machine-learning model may be a support vector machine. Support vector machines (i.e. support vector networks) are supervised learning models with associated learning algorithms that may be used to analyze data, e.g. in classification or regression analysis. Support vector machines may be trained by providing an input with a plurality of training input values that belong to one of two categories. The support vector machine may be trained to assign a new input value to one of the two categories. Alternatively, the machine-learning model may be a Bayesian network, which is a probabilistic directed acyclic graphical model. A Bayesian network may represent a set of random variables and their conditional dependencies using a directed acyclic graph. Alternatively, the machine-learning model may be based on a genetic algorithm, which is a search algorithm and heuristic technique that mimics the process of natural selection.

The following enumerated embodiments are disclosed.

Enumerated embodiment 1 is a vehicular device for determining occupant respiration, which includes at least one processor. The device is configured to receive sensor data and determine occupant respiration based on the sensor data. The device includes, a plurality of sensors that transmit sensor data to the at least one processor.

Enumerated embodiment 2 is the vehicular device of enumerated embodiment 1, in which the plurality of sensors includes at least one acoustic sensor. Enumerated embodiment 3 is the vehicular device of enumerated embodiment 2, wherein the acoustic sensor(s) includes a directional microphone which can be at an instrument panel or at a steering wheel.

Enumerated embodiment 4 is the vehicular device of enumerated embodiment 2 or 3, further including a noise filter configured for noise cancellation, the noise filter communicatively coupled to a plurality of acoustic sensors that includes the at least one acoustic sensor.

Enumerated embodiment 5 is the vehicular device of any preceding enumerated embodiment, wherein the plurality of sensors includes at least one camera. Enumerated embodiment 6 is the vehicular device of enumerated embodiment 5, in which the camera(s) is configured to determine at least one of thermal data or visible light data of a facial region of an occupant.

Enumerated embodiment 7 is the vehicular device of any preceding enumerated embodiment, in which occupant respiration includes at least one of: respiration rate; respiration amplitude; and respiration phase. The phase can include inhalation, exhalation, and possibly the transitions therebetween (e.g. from inhalation to exhalation or from exhalation to inhalation).

Enumerated embodiment 8 is the vehicular device of any preceding enumerated embodiment, in which the device, such as the at least one processor thereof, is configured for parallel execution of (i) classifying an audio signal based on audio sensor data as inhalation, exhalation, or ambience; and (ii) determining a transition of exhalation and inhalation.

Enumerated embodiment 9 is the vehicular device of any preceding enumerated embodiment, in which the device, such as the at least one processor thereof, determines a facial region of an occupant based on the visible light data, and optionally determines a bounding box based on the facial region. The bounding box can have a quaternion format.

Enumerated embodiment 10 is the vehicular device of any preceding enumerated embodiment, in which the device (such as the processor(s) thereof) is configured to determine a target direction of the directional microphone based on camera data from the facial region.

Enumerated embodiment 11 is the vehicular device of any preceding enumerated embodiment, configured to determine occupant respiration based on thermal camera data at the facial region.

Enumerated embodiment 12 is the vehicular device of any preceding enumerated embodiment, configured to execute sensor fusion machine learning, based on sensor fusion input, to determine the occupant respiration. The sensor fusion input can include acoustic data, thermal data, and/or visible light data.

Enumerated embodiment 13 is a method of determining vehicle occupant respiration, comprising acquiring sensor data from a plurality of sensors in a vehicle, and determining occupant respiration based on the sensor data.

Enumerated embodiment 14 is the method of enumerated embodiment 13, wherein determining occupant respiration includes: determining at least one of: respiration rate; respiration amplitude; and respiration phase. Phase can include inhalation, exhalation, and possibly the transitions therebetween.

Enumerated embodiment 15 is the method of enumerated embodiment 13 or 14, further comprising: classifying an audio signal based on audio sensor data of the sensor data as inhalation, exhalation, or ambience; and determining a transition of exhalation and inhalation. The classifying and determining the transition can be parallelly determined, such as by a multithread processor.

Enumerated embodiment 16 is the method of any of one of enumerated embodiments 13-15, also including determining a facial region of an occupant based on visible light data of the sensor data.

Enumerated embodiment 17 is the method of enumerated embodiment 16, further comprising determining a bounding box based on the facial region. The bounding box can have a quaternion format.

Enumerated embodiment 18 is the method of enumerated embodiment 16 or 17, further comprising determining occupant respiration based on thermal camera data at the facial region.

Enumerated embodiment 19 is the method of any one of enumerated embodiments 13-18, further comprising: executing sensor fusion machine learning to determine the occupant respiration based on sensor fusion input. The sensor fusion input can include at least one of: acoustic data, thermal data, or visible light data.

Enumerated embodiment 20 is a non-transitory computer readable medium including instructions adapted to determine vehicle occupant respiration, comprising: acquiring sensor data from a plurality of sensors in a vehicle, and determining occupant respiration based on the sensor data.

The following claims are hereby incorporated in the detailed description, wherein each claim may stand on its own as a separate example. It should also be noted that although in the claims a dependent claim refers to a particular combination with one or more other claims, other examples may also include a combination of the dependent claim with the subject matter of any other dependent or independent claim. Such combinations are hereby explicitly proposed, unless it is stated in the individual case that a particular combination is not intended. Furthermore, features of a claim should also be included for any other independent claim, even if that claim is not directly defined as dependent on that other independent claim.

What is claimed is:

1. A vehicular device for determining occupant respiration, comprising:
at least one processor configured to receive sensor data and determine occupant respiration based on the sensor data, and
a plurality of sensors configured to transmit sensor data to the at least one processor.

2. The vehicular device of claim 1, wherein
the plurality of sensors includes at least one acoustic sensor.

3. The vehicular device of claim 2, wherein
the at least one acoustic sensor includes a directional microphone at an instrument panel or at a steering wheel.

4. The vehicular device of claim 2, further comprising:
a noise filter configured for noise cancellation, the noise filter communicatively coupled to a plurality of acoustic sensors that includes the at least one acoustic sensor.

5. The vehicular device of claim 1, wherein
the plurality of sensors includes at least one camera.

6. The vehicular device of claim 5, wherein
the at least one camera is configured to determine at least one of thermal data or visible light data of a facial region of an occupant.

7. The vehicular device of claim 1, wherein
occupant respiration includes at least one of: respiration rate; respiration amplitude;
and respiration phase which includes inhalation, exhalation, and transitions therebetween.

8. The vehicular device of claim 1, wherein
the at least one processor is configured for:
parallel execution of:
classifying an audio signal based on audio sensor data as inhalation, exhalation, or ambience; and
determining a transition of exhalation and inhalation.

9. The vehicular device of claim 6, wherein
the at least one processor is configured for:
determining a facial region of an occupant based on the visible light data, and
determining a bounding box based on the facial region, the bounding box having a quaternion format.

10. The vehicular device of claim 9, wherein
the at least one processor is configured for:
determining a target direction of a directional microphone based on camera data from the facial region.

11. The vehicular device of claim 1, wherein
the at least one processor is configured for:
determining occupant respiration based on thermal camera data at a facial region.

12. The vehicular device of claim 1, wherein
the at least one processor is configured for:
executing sensor fusion machine learning, based on sensor fusion input, to determine the occupant respiration; wherein
sensor fusion input includes at least one of: acoustic data, thermal data, or visible light data.

13. A method of determining vehicle occupant respiration, comprising:
acquiring sensor data from a plurality of sensors in a vehicle, and
determining occupant respiration based on the sensor data.

14. The method of claim 13, wherein
determining occupant respiration includes:
determining at least one of: respiration rate; respiration amplitude; and respiration phase which includes inhalation, exhalation, and transitions therebetween.

15. The method of claim 13, further comprising:
classifying an audio signal based on audio sensor data of the sensor data as inhalation, exhalation, or ambience; and
determining a transition of exhalation and inhalation; wherein
the classifying and determining the transition are parallelly determined by a multithread processor.

16. The method of claim 13, further comprising:
determining a facial region of an occupant based on visible light data of the sensor data.

17. The method of claim 16, further comprising:
determining a bounding box based on the facial region, the bounding box having a quaternion format.

18. The method of claim 16, further comprising:
determining occupant respiration based on thermal camera data at the facial region.

19. The method of claim 13, further comprising:
executing sensor fusion machine learning to determine the occupant respiration based on sensor fusion input; wherein
the sensor fusion input includes at least one of: acoustic data, thermal data, or visible light data.

20. A non-transitory computer readable medium including instructions adapted to determine vehicle occupant respiration, comprising:
acquiring sensor data from a plurality of sensors in a vehicle, and
determining occupant respiration based on the sensor data.
